(19) United States
(12) Patent Application Publication
(10) Pub. No.: US 2010/0291275 A1
(43) Pub. Date: Nov. 18, 2010

(54) Flavorant and Fragrance Furan-2(5H)-One Compounds

(76) Inventors: Stefan Michael Furrer, Cincinnati, OH (US); Peter Schieberle, Freising (DE); Andrea Burdack-Freitag, Holzkirchen (DE)

Correspondence Address: CURATOLO SIDOTI CO., LPA, 24500 CENTER RIDGE ROAD, SUITE 280, CLEVELAND, OH, 44145, US

(21) Appl. No.: 12/293,591

(22) PCT Filed: Mar. 15, 2007
(86) PCT No.: PCT/CH07/00142

§ 371 Date: Oct. 28, 2008

(51) Int. Cl. A23L 1/22; C07D 307/58

(52) U.S. Cl. 426/442; 549/295

(57) ABSTRACT

A method of providing a flavour or a fragrance to a composition, which includes adding to the composition a compound of the formula I

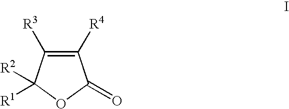

in which R¹ and R³ are independently selected from at least one of H, C₁-C₁₀ straight chain alkyl or branched chain alkyl, and R² and R⁴ are independently selected from at least one of C₁-C₁₀ straight chain alkyl or branched chain alkyl. Flavored and fragranced products including a compound of the Formula I are also provided.

This invention relates to flavorants and fragrances and compounds for use therein.

A method of providing a flavour or a fragrance to a composition, comprising the addition thereto of a compound of the formula I

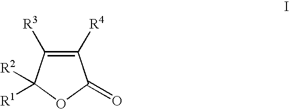

in which R¹ and R³ are independently selected from at least one of H, C₁-C₁₀ straight chain alkyl or branched chain alkyl, and in which R² and R⁴ are independently selected from at least one of C₁-C₁₀ straight chain alkyl or branched chain alkyl.

According to certain illustrative embodiments, the compound of Formula I include those wherein R¹, R², R³, and R⁴ are represented by the following:

R¹ is C₁-C₁₀ straight or branched chain alkyl;

R² is C₁-C₁₀ straight or branched chain alkyl or R¹ and R² together form a 3-8-membered ring;

R³ is H, C₁-C₁₀ straight or branched chain alkyl;

R⁴ is C₁-C₁₀ straight or branched chain alkyl.

According to further illustrative embodiments, the compound of Formula I include those wherein R¹, R², R³, and R⁴ are represented by the following:

R¹ is at least one of methyl or ethyl;

R² is at least one C₁-C₁₀ straight or branched chain alkyl or R¹ and R² together form a 3-8-membered ring;

R³ is at least one of H, methyl, or ethyl;

R⁴ is at least one of methyl, ethyl, propyl, butyl, pentyl, isopropyl, isobutyl or isopentyl.

The compounds of Formula (I) may comprise one or more chiral centres and as such may exist as a mixture of stereoisomers. Alternatively, the mixture of stereoisomers may be resolved as isomerically pure forms. As resolving stereoisomers adds to the complexity of manufacture and purification of these compounds, it is simply more economical to use the compounds as mixtures of their stereoisomers. If it is desired to prepare individual stereoisomers, this may be achieved according to methods known in the art, for example, preparative HPLC and GC or by stereoselective syntheses.

While certain of the compounds are known, for example, 3,5,5-trimethyl-2(5H)-furanone is known in green tea (see, for example, Shimoda et al, J. Agr. Food. Chem. (1995) 43(6), 1621-5), others are novel compounds not previously disclosed. Thus, in other aspects, provided are compounds of Formula (I) selected from those in which the groups R¹, R², R³ and R⁴ in illustrative individual compounds are selected as follows:

| R¹ | R² | R³ | R⁴ |
|---|---|---|---|
| Methyl | heptyl | hydrogen | methyl |
| Methyl | Octyl | hydrogen | methyl |
| Methyl | Nonyl | hydrogen | methyl |
| Methyl | Decyl | hydrogen | methyl |
| Methyl | isopentyl | hydrogen | methyl |
| Ethyl | Ethyl | hydrogen | methyl |
| Ethyl | Propyl | hydrogen | methyl |
| Ethyl | Pentyl | hydrogen | methyl |
| Ethyl | Hexyl | hydrogen | methyl |
| Ethyl | Heptyl | hydrogen | methyl |
| Ethyl | Octyl | hydrogen | methyl |
| Ethyl | Nonyl | hydrogen | methyl |
| Ethyl | Decyl | hydrogen | methyl |

| | | |
|---|---|---|
| 3-membered ring with R2 | hydrogen | methyl |
| 4-membered ring with R2 | hydrogen | methyl |
| 5-membered ring with R2 | hydrogen | methyl |
| 7-membered ring with R2 | hydrogen | methyl |

| | | | |
|---|---|---|---|
| Methyl | Hexyl | methyl | methyl |
| Methyl | Heptyl | methyl | methyl |
| Methyl | Octyl | methyl | methyl |
| Methyl | Nonyl | methyl | methyl |
| Methyl | Decyl | methyl | methyl |
| Methyl | Methyl | hydrogen | Pentyl |
| Methyl | Methyl | hydrogen | Hexyl |
| Methyl | Methyl | hydrogen | Heptyl |
| Methyl | Methyl | hydrogen | Octyl |
| Methyl | Methyl | hydrogen | Nonyl |
| Methyl | Methyl | hydrogen | Decyl |

The compounds may be obtained from natural sources or synthesised by known methods, for example, by that described by Iwahama et al in Chem. Comm., 2000, 613-614 and Yates in Can. J. Chem., 1987, 68 (8), 1695, both of which are incorporated herein by reference in their entireties.

The compounds of Formula I are useful for the provision of flavours in compositions that are to be taken orally. Thus, in certain embodiments, a composition for oral reception or ingestion is provided, the composition comprising a flavorant comprising a compound of Formula I as hereinabove described. The composition to which the compound of Formula I is added may be any such composition. For example, it may be a foodstuff, baked goods, confectionery, including chewing gum and hard candy, a beverage, a tobacco product, a dental preparation, such as a toothpaste, a tooth gel or a mouthwash, or a solid, liquid or sprayable medicinal preparation. Therefore, also provided is a flavoured product that is orally receivable or ingestible, in which the flavoured product comprises a flavorant that is a compound according to Formula I above.

The compounds of Formula I are also useful as fragrances. They may be used in fine fragrances, such as perfumes, or as functional fragrances in any of the wide range of products that contain fragrance, for example, detergents, soaps and other personal products, such as creams and lotions, body care products, cosmetic products, household cleaning agents and preparations, polishes, consumer healthcare products, air fresheners and the like. Therefore, further provided is a fragranced product, in which the fragranced product comprises a fragrance that is a compound according to Formula I above.

The phrases “a compound of Formula I” and “a compound according to Formula I” refer to embodiments in which one such compound, or a mixture or combination of more than one such compound, is used.

The compounds of Formula I may be added to products in the conventional manner using standard equipment and in generally art-recognised proportions. These will naturally vary, depending on the use and extent of flavour or fragrance desired. It is also possible to exceed the generally-recognised proportions, should a particular effect should be desired. In addition, such products may also incorporate the particular raw materials known to the art for use in that particular product, again in the usual proportions.

The method, compounds, flavoured compositions, and fragranced compositions are further described with reference to the following non-limiting, illustrative examples.
3,5,5-trimethylfuran-2(5H)-one
5-ethyl-3,5-dimethylfuran-2(5H)-one
5-butyl-3,5-dimethylfuran-2(5H)-one
5-hexyl-3,5-dimethylfuran-2(5H)-one
5-isopentyl-3,5-dimethylfuran-2(5H)-one
3,5-dimethyl-5-octylfuran-2(5H)-one
5-decyl-3,5-dimethylfuran-2(5H)-one
3-ethyl-5,5-dimethylfuran-2(5H)-one
3,4,5,5-tetramethylfuran-2(5H)-one
3-butyl-5,5-dimethylfuran-2(5H)-one
3-hexyl-5,5-dimethylfuran-2(5H)-one
5,5-diethyl-3-methylfuran-2(5H)-one
3-methyl-1-oxaspiro[4.5]dec-3-en-2-one
3-methyl-1-Oxaspiro[4.4]non-3-en-2-one

Certain exemplary compounds and their characteristics are shown below, followed by the method of preparation:

| | Example | |
|---|---|---|
| Taste description (at 10 ppm in water): nutty, brown, condensed milk, nutmeat, slight fatty mouthfeel, and added slight astringent aftertaste. | 1 | 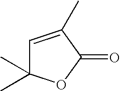 |
| Taste description (at 1 ppm in water): Mushroom, musty, oily mouthfeel, greasy, and added astringent aftertaste. | 2 | 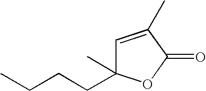 |
| Odour Description: linear, celery leaves, clean, linear, natural, fenugreek. | 3 | 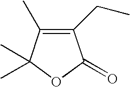 |
| Odour Description: slightly minty, green, fruity, buttery. | 4 | 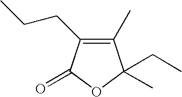 |
| Odour Description: buttery, creamy, celery, fatty. | 5 | 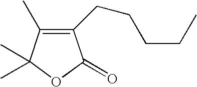 |

Methods for the general preparation of unsaturated lactones from α-hydroxy-γ-lactones are described in T. Iwahama, S. Sakaguchi, Y. Ishii, Chem. Comm., 2000, 613-614 and P. Yates, Can. J. Chem., 1987, 68 (8), 1695.

### Example 1

### 3,5,5-trimethylfuran-2(5H)-one

A) Preparation of dihydro-3-hydroxy-3,5,5-trimethylfuran-2(3H)-one

In a 500 mL flask, fitted with magnetic stirrer, reflux condenser, 3.26 g of N-hydroxyphtalimide, 120 g of isopropanol, 0.05 g of Cobalt (II) acetate (4H₂O) and 0.71 g of cobalt (III)acetylacetonate and 33 mL of acetonitrile were added. The flask was evacuated and placed under an oxygen atmosphere (balloon). 20 g of methyl methacrylate were added at room temperature and the greenish solution was heated to 70° C. and stirred at 70° C. for 6 h and for 16 h (over night) at RT (under nitrogen), while it became brownish.

The mixture was concentrated and purified by column chromatography and crystallization from MTBE/Hexanc, yielding 5.63 g white crystals (19.5% yield).

¹H NMR (300 MHz; CDCl₃) δ: 2.75 (s, 1H) 2.32 (d, 1H), 2.10 (d, 1H), 1.53 (d, 6H), 1.46 (s, 3H)

¹³C NMR (300 MHz; CDCl₃) δ: 178.49, 82.21, 48.45, 29.13, 28.98, 26.30

mp: 66-68° C.

B) Preparation of 3,5,5-trimethylfuran-2(5H)-one

In a 100 mL flask, fitted with magnetic stirrer, reflux condenser, 5.00 g of dihydro-3-hydroxy-3,5,5-trimethylfuran-2(3H)-one and 29.2 g of hydrobromic acid (48% in water) were added and heated at reflux (135° C. oilbath) for 4 h. The mixture was poured on ice and extracted with MTBE. The organic layer was washed with brine, dried over magnesium sulfate and concentrated. The crude product was crystallized from MTBE and washed with hexane, yielding 1.75 g white crystals (40% yield).

¹H NMR (300 MHz; CDCl₃) δ: 7.00 (dd, 1H), 1.89 (d, 3H), 1.44 (s, 6H)

¹³C NMR (300 MHz; CDCl₃) δ: 173.63, 153.92, 128.18, 84.06, 25.63, 10.41

MS: 126 (M+), 111, 83, 67, 55, 43, 39

IR: 3462.2, 3087.9, 2983.0, 2932.1, 1744.3, 1665.7

mp: 54-55° C.

### Example 2

### 5-butyl-3,5-dimethylfuran-2(5H)-one

5-butyl-3,5-dimethylfuran-2(5H)-one was prepared according to the general procedure in Example 1, using 2-hexanol and methyl methacrylate.

¹H NMR (300 MHz; CDCl₃) δ: 6.9 (dd, 1H), 1.90 (d, 3H), 1.8-1.6 (m, 2H), 1.41 (s, 3H), 1.3-1.2 (m, 4H), 0.9 (t, 3H)

¹³C NMR (300 MHz; CDCl₃) δ: 173.83, 153.06, 128.75, 86.47, 38.36, 24.14, 22.73, 13.83, 10.47

MS: 168 (M+), 153, 140, 125, 111, 97, 83, 69, 55, 43

### Example 3

### 3-ethyl-4,5,5-trimethylfuran-2(51-1)-one

In a round bottom flask, 13.8 g of potassium carbonate (0.1 mol), 18.8 g of Diethyl ethylmalonate (0.1 mol) and 10.2 g of 3-Hydroxy-3-methyl-2-butanone (0.1 mol) were added and heated for 6 h at 180° C. MTBE was added to the reaction mixture, the reaction mixture was filtered and concentrated. The crude mixture was distilled by Kugelrohr distillation, yielding 10.0 g (64% yield).

¹H NMR (200 MHz; CDCl₃) δ: 2.3 (dd, 2H), 1.9 (s, 3H), 1.4 (s, 6H), 1.1 (t, 3H)

### Example 4

### 5-ethyl-4,5-dimethyl-3-propylfuran-2(5H)-one

5-ethyl-4,5-dimethyl-3-propylfuran-2(5H)-one was prepared according to example 3, using 3-Hydroxy-3-methyl-2-pentanone and Diethyl propylmalonate.

¹H NMR (200 MHz; CDCl₃) δ: 2.2 (t, 2H), 2.0-1.8 (m, 2H), 1.9 (s, 3H), 1.7-1.4 (m, 4H), 1.4 (s, 3H), 0.9 (t, 3H), 0.8 (t, 3H)

### Example 5

### 4,5,5-trimethyl-3-pentylfuran-2(5H)-one

4,5,5-timethyl-3-pentylfuran-2(5H)-one was prepared according to example 3, using 3-Hydroxy-3-methyl-2-butanone and Diethyl pentylmalonate.

¹H NMR (200 MHz; CDCl₃) δ: 2.2 (t, 2H), 1.9 (s, 3H), 1.6-1.4 (m, 2H), 1.4 (s, 3H), 1.4-1.2 (m, 4H), 0.9 (t, 3H)

### Example 6

### Hazelnut

10 ppm of 3,5,5-trimethylfuran-2(5H)-one (Example 1) was added to a beverage base (10 g sugar, 90 g water) containing 0.65% by weight of hazelnut fluid extract (a hydroalcoholic extract (ethanol and propylene glycol) of hazelnut, corylus Americana, ex Chart). The resulting composition showed a fuller, toasted, slight roasted, brown, slightly creamy, definitely nutty, slight macadamia nut aroma, while the hazelnut extract alone was slightly sweet, woody, astringent and rather plain at that level.

The phrase “at least one of” as used herein means one member of a given group or listing of members, or combinations of two or more members of the given group of members. The phrase “at least one of” does not require one of each member of a given group of members.

While the method, compound, flavoured composition, and fragranced composition have been described above in connection with certain illustrative embodiments, it is to be understood that other similar embodiments may be used or modifications and additions may be made to the described embodiments. Furthermore, all embodiments disclosed are not necessarily in the alternative, as various embodiments may be combined to provide the desired characteristics. Variations can be made by one having ordinary skill in the art without departing from the spirit and scope of the invention. Therefore, method, compound, flavoured composition, and fragranced composition should not be limited to any single embodiment, but rather construed in breadth and scope in accordance with the recitation of the attached claims.

1. A method of providing a flavour or a fragrance to a composition, comprising adding to said composition a compound of the Formula I 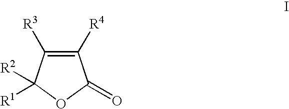
wherein R¹ and R³ are independently selected from at least one of H, C₁-C₁₀ straight chain alkyl or C₃-C₁₀ branched chain alkyl, and wherein R² and R⁴ are independently selected from at least one of C₁-C₁₀ straight or C₃-C₁₀ branched chain alkyl.

2. The method according to claim 1, wherein each of R¹, R², R³ and R⁴ of Formula I are independently selected from the following:
(a) R¹ is at least one of C₁-C₁₀ straight chain alkyl or C₃-C₁₀ branched chain alkyl;
(b) R² is at least one of C₁-C₁₀ straight chain alkyl or C₃-C₁₀ branched chain alkyl, or R¹ and R² together form a 3-8-membered ring;
(c) R³ is at least one of H, C₁-C₁₀ straight or C₃-C₁₀ branched chain alkyl; and
(d) R⁴ is at least one of C₁-C₁₀ straight chain alkyl or C₃-C₁₀ branched chain alkyl.

3. The method according to claim 2, wherein each of R¹, R², R³ and R⁴ of Formula I are independently selected from at least one of the following:
(a) R¹ is selected from at least one of methyl, ethyl, isopropyl or isobutyl;
(b) R² is selected from at least one of C₁-C₁₀ straight chain alkyl or C₃-C₁₀ branched chain alkyl, or R¹ and R² together form a 3-8 membered ring;
(c) R³ is selected from at least one of H, methyl, ethyl, isopropyl or isobutyl; and
(d) R⁴ is selected from at least one of methyl, ethyl, propyl, butyl, pentyl, isopropyl, isobutyl or isopentyl.

4. The method according to claim 3, wherein each of R¹, R², R³ and R⁴ of Formula I are independently selected from at least one of the following:
(a) R¹ is selected from at least one of methyl or ethyl;
(b) R² is selected from at least one of C₁-C₁₀ straight chain alkyl or C₃-C₁₀ branched chain alkyl, or R¹ and R² together form a 3-8 membered ring;
(c) R³ is selected from at least one of H, methyl or ethyl; and
(d) R⁴ is selected from at least one of methyl, ethyl, propyl, butyl, pentyl, isopropyl, or isobutyl.

5. A compound comprising Formula I 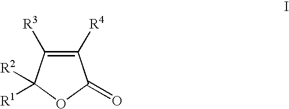
wherein R¹, R², R³ and R⁴ for individual compounds are selected as follows:

| R¹ | R² | R³ | R⁴ |
|---|---|---|---|
| Methyl | heptyl | H | methyl |
| Methyl | Octyl | H | methyl |
| Methyl | Nonyl | H | methyl |
| Methyl | Decyl | H | methyl |
| Methyl | isopentyl | H | methyl |
| Ethyl | Ethyl | H | methyl |
| Ethyl | Propyl | H | methyl |
| Ethyl | Pentyl | H | methyl |
| Ethyl | Hexyl | H | methyl |
| Ethyl | Heptyl | H | methyl |
| Ethyl | Octyl | H | methyl |
| Ethyl | Nonyl | H | methyl |
| Ethyl | Decyl | H | methyl |
| | | |
|---|---|---|
| 3-membered ring with R2 | H | methyl |
| 4-membered ring with R2 | H | methyl |
| 5-membered ring with R2 | H | methyl |
| 7-membered ring with R2 | H | methyl |
| | | | |
|---|---|---|---|
| Methyl | Hexyl | methyl | methyl |
| Methyl | Heptyl | methyl | methyl |
| Methyl | Octyl | methyl | methyl |
| Methyl | Nonyl | methyl | methyl |
| Methyl | Decyl | methyl | methyl |
| Methyl | Methyl | H | Pentyl |
| Methyl | Methyl | H | Hexyl |
| Methyl | Methyl | H | Heptyl |
| Methyl | Methyl | H | Octyl |
| Methyl | Methyl | H | Nonyl |
| Methyl | Methyl | H | Decyl |

6. A flavored product that is orally receivable or ingestible, comprising a flavorant that is a compound of Formula I: 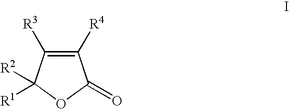
wherein R¹ and R³ are independently selected from at least one of H, C₁-C₁₀ straight chain alkyl or C₃-C₁₀ branched chain alkyl, and wherein R² and R⁴ are independently selected from at least one of C₁-C₁₀ straight chain alkyl or C₃-C₁₀ branched chain alkyl.

7. The flavoured product according to claim 6, wherein each of R¹, R², R³ and R⁴ of Formula I are independently selected from the following:
(a) R¹ is at least one of C₁-C₁₀ straight chain alkyl or C₃-C₁₀ branched chain alkyl:
(b) R² is at least one of C₁-C₁₀ straight chain alkyl or C₃-C₁₀ branched chain alkyl, or R¹ and R² together form a 3-8-membered ring:
(c) R³ is at least one of H, C₁-C₁₀ straight or C₃-C₁₀ branched chain alkyl; and
(d) R⁴ is at least one of C₁-C₁₀ straight chain alkyl or C₃-C₁₀ branched chain alkyl.

8. The flavoured product according to claim 7, wherein each of R¹, R², R³ and R⁴ of Formula I are independently selected from at least one of the following:
(a) R¹ is selected from at least one of methyl, ethyl, isopropyl or isobutyl;
(b) R² is selected from at least one of C₁-C₁₀ straight chain alkyl or C₃-C₁₀ branched chain alkyl, or R¹ and R² together form a 3-8 membered ring;
(c) R³ is selected from at least one of H, methyl, ethyl, isopropyl or isobutyl; and
(d) R⁴ is selected from at least one of methyl, ethyl, propyl, butyl, pentyl, isopropyl, isobutyl or isopentyl.

9. The flavoured product according to claim 8, wherein each of R¹, R², R³ and R⁴ of Formula I are independently selected from at least one of the following:
(a) R¹ is selected from at least one of methyl or ethyl;
(b) R² is selected from at least one of C₁-C₁₀ straight chain alkyl or C₃-C₁₀ branched chain alkyl, or R¹ and R² together form a 3-8 membered ring:
(c) R³ is selected from at least one of H, methyl or ethyl; and
(d) R⁴ is selected from at least one of methyl, ethyl, propyl, butyl, pentyl, isopropyl, or isobutyl.

10. A fragranced product, comprising a fragrance that is a compound of Formula I: 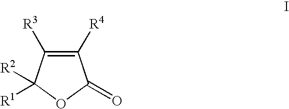
wherein R¹ and R³ are independently selected from at least one of H, C₁-C₁₀ straight chain alkyl or C₃-C₁₀ branched chain alkyl, and wherein R² and R⁴ are independently selected from at least one of C₁-C₁₀ straight chain alkyl or C₃-C₁₀ branched chain alkyl.

11. The fragranced product according to claim 10, wherein each of R¹, R², R³ and R⁴ of Formula I are independently selected from the following:
(a) R¹ is at least one of C₁-C₁₀ straight chain alkyl or C₃-C₁₀ branched chain alkyl;
(b) R² is at least one of C₁-C₁₀ straight chain alkyl or C₃-C₁₀ branched chain alkyl, or R¹ and R² together form a 3-8-membered ring;
(c) R³ is at least one of 1-1, C₁-C₁₀ straight or C₃-C₁₀ branched chain alkyl; and
(d) R⁴ is at least one of C₁-C₁₀ straight chain alkyl or C₃-C₁₀ branched chain alkyl.

12. The fragranced product according to claim 11, wherein each of R¹, R², R³ and R⁴ of Formula I are independently selected from at least one of the following:
(a) R¹ is selected from at least one of methyl, ethyl, isopropyl or isobutyl;
(b) R² is selected from at least one of C₁-C₁₀ straight chain alkyl or C₃-C₁₀ branched chain alkyl, or R¹ and R² together form a 3-8 membered ring;
(c) R³ is selected from at least one of H, methyl, ethyl, isopropyl or isobutyl; and
(d) R⁴ is selected from at least one of methyl, ethyl, propyl, butyl, pentyl, isopropyl, isobutyl or isopentyl.

13. The fragranced product according to claim 12, wherein each of R¹, R², R³ and R⁴ of Formula I are independently selected from at least one of the following:
(a) R¹ is selected from at least one of methyl or ethyl;
(b) R² is selected from at least one of C₁-C₁₀ straight chain alkyl or C₃-C₁₀ branched chain alkyl, or R¹ and R² together form a 3-8 membered ring;
(c) R³ is selected from at least one of H, methyl or ethyl; and
(d) R⁴ is selected from at least one of methyl, ethyl, propyl, butyl, pentyl, isopropyl, or isobutyl.
